(19) United States
(12) Patent Application Publication
(10) Pub. No.: US 2026/0269056 A1
(43) Pub. Date: Sep. 10, 2026

(54) OPTICAL IMAGING DISTORTION COMPENSATION

(71) Applicant: ALCON INC., Fribourg (CH)

(72) Inventors: Dan Teodorescu, Irvine, CA (US); Mikhail Ovchinnikov, Dana Point, CA (US); Marc Nestor, Aliso Viejo, CA (US)

(21) Appl. No.: 19/327,122

(22) Filed: Sep. 12, 2025

Related U.S. Application Data
(60) Provisional application No. 63/695,883, filed on Sep. 18, 2024.

(51) Int. Cl. G16H 40/63; A61F 9/008; G06T 5/80; G06T 7/00; G06V 10/44; G06V 20/50

(52) U.S. Cl. CPC G16H 40/63; A61F 9/008; G06T 5/80; G06T 7/0012; G06V 10/44; G06V 20/50; A61F 2009/00851; G06T 2207/10101; G06T 2207/30041; G06V 2201/03

(57) ABSTRACT

The present disclosure relates to operations that include identifying, based on an optical imaging result corresponding to a tissue, a non-uniform layer that is disposed in an optical measurement path corresponding to the optical imaging result. The operations may also include performing one or more adjustment operations with respect to the optical imaging result based on identification of the non-uniform layer.

### CROSS-REFERENCE TO RELATED APPLICATION

This application claims priority to U.S. Provisional Patent Application No. 63/695,883, filed on Sep. 18, 2024, the disclosure of which is incorporated herein by reference in its entirety.

### BACKGROUND

Optical imaging (e.g., Optical Coherence Tomography (OCT)) is often used in ophthalmology to obtain images corresponding to the eye. For example, ophthalmology surgical systems may utilize optical imaging to help facilitate the tasks they perform.

### SUMMARY

The present disclosure relates to operations that include identifying, based on an optical imaging result corresponding to a tissue, a non-uniform layer that is disposed in an optical measurement path corresponding to the optical imaging result. The operations may also include performing one or more adjustment operations with respect to the optical imaging result based on identification of the non-uniform layer.

### BRIEF DESCRIPTION OF THE DRAWINGS

The present disclosure relates to systems and methods for performing distortion compensation with respect to optical imaging, wherein:

FIG. 1A is a block diagram of an example ophthalmic surgical system that may be used with and/or implement one or more embodiments of the present disclosure related to optical imaging distortion compensation;

FIG. 1B illustrates an example of measurement light passing through a first lens, a liquid layer, and a second lens prior to interacting with a target tissue, according to one or more embodiments of the present disclosure;

FIG. 1C illustrates a cross-sectional view of an example soft-fit insert, according to one or more embodiments of the present disclosure;

FIG. 2 is a flow diagram illustrating a method 200 for compensating for optical imaging distortion, according to one or more embodiments of the present disclosure; and

FIG. 3 is a block diagram of an example computing system suitable for use in implementing one or more embodiments of the present disclosure.

### DETAILED DESCRIPTION

In ophthalmology, optical imaging may be used to generate images and/or representations of different parts of the eye—e.g., the retina, optic nerve, and anterior segment of the eye. In some embodiments, the optical imaging may include Optical Coherence Tomography (OCT). OCT operates on the principle of low-coherence interferometry, utilizing light waves in a manner similar to how ultrasound uses sound waves. A light source, such as a superluminescent diode or a laser, emits a beam that is split into two paths: one directed at the tissue being imaged and the other toward a reference mirror. The light that reflects back from the tissue and the reference mirror is recombined to create an interference pattern, which is analyzed to generate depth information about the tissue. By scanning the light across the tissue, a two-dimensional or three-dimensional image is constructed. As the light penetrates the tissue, it reflects differently based on the varying densities and structures within the tissue. These differences in reflections are captured and processed to form detailed images of the internal microstructures.

In some instances, an optical measurement path of the optical imaging—e.g., the path of measurement light (e.g., the directed light and/or the reflected light of OCT)—may include elements that may cause distortion in the images that are generated based on such measurement light. For example, in some instances, a liquid layer may be disposed in the optical measurement path. The liquid layer may have certain refractive and/or reflective properties that may distort the measurement light as it travels along its path. Additionally or alternatively, the liquid layer may be non-uniform in nature and/or may be changing over time such that the effects on the measurement light may be inconsistent at different parts of the liquid layer and/or may change. Such effects on the measurement light may cause distortions in the images of the tissue (e.g., the eye) that may be generated therefrom.

One or more embodiments of the present disclosure relate to compensating for distortion in optical imaging (e.g., Optical Coherence Tomography (OCT)) related to ophthalmic applications. In particular, in some embodiments, a liquid layer may be identified from an optical imaging result corresponding to a tissue—e.g., a cross-sectional image of the tissue. In these and other embodiments, one or more adjustment operations may be performed with respect to the optical imaging result. The adjustment operations may correspond to accounting for distortion in the optical imaging result that may be caused by the liquid layer.

The embodiments of the present disclosure may be utilized with any suitable system, apparatus or device in which such distortion compensation may be beneficial. For example, in some embodiments an ophthalmic surgical system may be configured to perform optical imaging and may be configured to also perform distortion compensation with respect to the optical imaging. Such compensation may allow for the ophthalmic surgical system to utilize more accurate images of the eye in the performance of its operations, which may accordingly result in an improvement in such operations. Therefore, one or more embodiments of the present disclosure may improve ophthalmic surgical systems by improving the accuracy in the imaging used and the operations performed based on such imaging. Such embodiments may accordingly cause better accuracy and/or precision in the operations performed, which may improve the efficacy and/or safety of such ophthalmic surgical systems.

By way of a particular example, femto-second laser assisted cataract surgery (FLACS) is an advanced technique in cataract surgery that uses a femtosecond laser to assist in various steps of the procedure. For example, the femtosecond laser emits ultra-short pulses of light (on the order of 10-15 seconds) to make precise cuts and create openings in the eye. This technology enhances the accuracy and safety of cataract surgery, improving outcomes and patient satisfaction.

For example, during femto-second laser assisted cataract surgery, the patient's eye may be imaged using an integrated OCT system. This imaging may guide the placement of the laser used to generate precise incisions and lens fragmentation as part of the surgery.

Further, a patient interface of the femto-second surgical system may include a soft-fit insert that is similar to a contact lens. In order to help maintain flexibility of the insert, the insert may be moistened—e.g., with a saline solution. The liquid layer that is created may be non-uniform in nature and may cause distortions in the imaging used to guide the laser placement. Additionally or alternatively, the soft-fit insert itself may have non-uniform thickness that may be cause different degrees of reflection and/or refraction that may also cause different levels of distortion. One or more embodiments of the present disclosure may be used to at least partially compensate for such distortion, which may result in more accurate placement of the laser. However, it is understood that such a result is only an example improvement that may be accomplished by compensating for such distortion.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to performance of distortion compensation in the context of ophthalmic surgical systems. However, such uses are not meant to be limiting such that the distortion compensation described may be used in any number of different contexts and applications where it may be helpful or applicable.

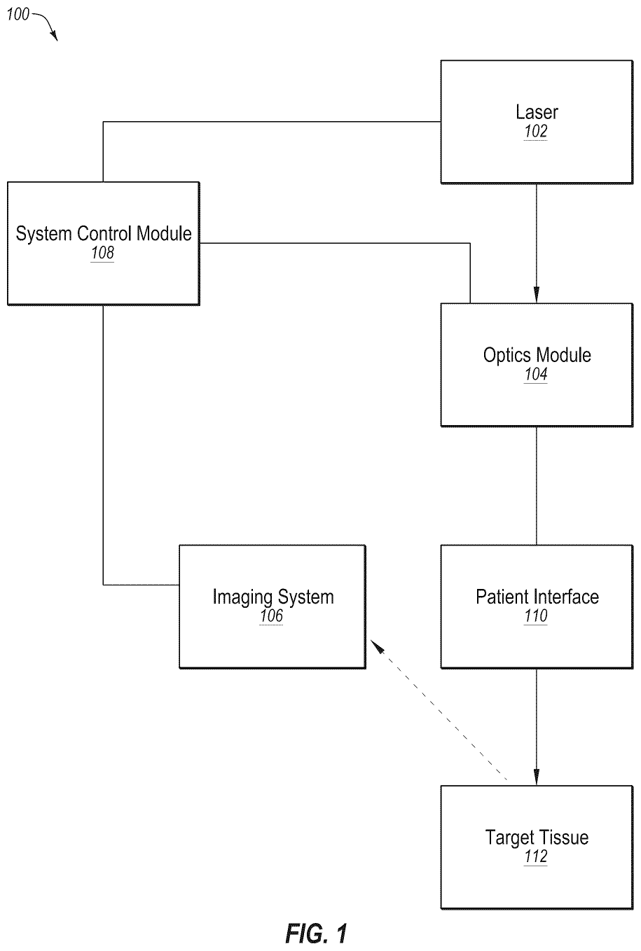

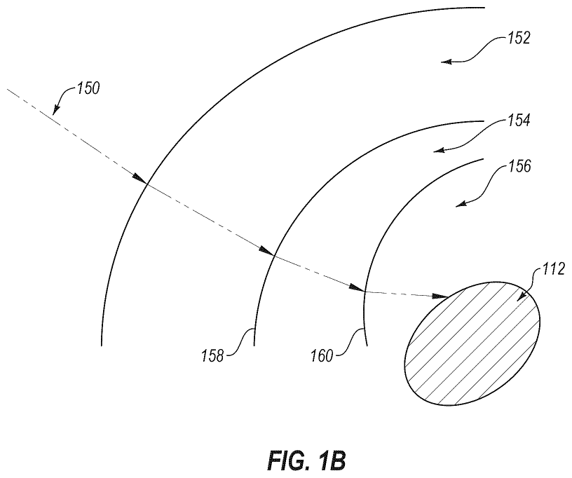

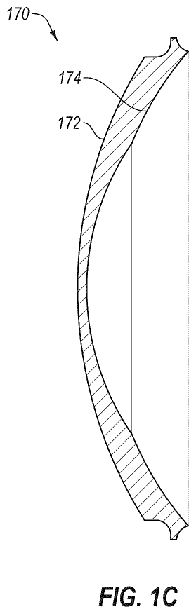

FIG. 1A is a block diagram of an example ophthalmic surgical system 100 (“system 100”) that may be used with and/or implement one or more embodiments of the present disclosure related to optical imaging distortion compensation. In general, the system 100 may be configured to perform one or more ophthalmic treatment operations with respect performance of a procedure corresponding to a target tissue 112, which may include a portion of an eye in some embodiments. In some embodiments, the system 100 may include a laser 102, an optics module 104, an imaging system 106, a system control module 108, and a patient interface 110.

The laser 102 may include any suitable system, apparatus, or device, configured to generate one or more laser beams that may be used to perform ophthalmic operations or tasks with respect to the target tissue 112. In some embodiments, the laser 102 may include multiple lasers that each generate an individual laser beam. Additionally or alternatively, the laser 102 may include a single laser that is configured to generate a single beam or multiple beams.

In some embodiments, the laser 102 may be configured to generate a pulsed laser beam that is pulsed at a high repetition rate at a pulse repetition rate of thousands of shots per second or higher with relatively low energy per pulse. For example, in some embodiments, the laser 102 may be a femtosecond laser that emits ultra-short pulses of light (e.g., on the order of 10-15 seconds). Such a laser may be operated to use a relatively low energy per pulse to localize the tissue effect of the target tissue 112 that may be caused by laser-induced photodisruption by the beam generated by the laser 102.

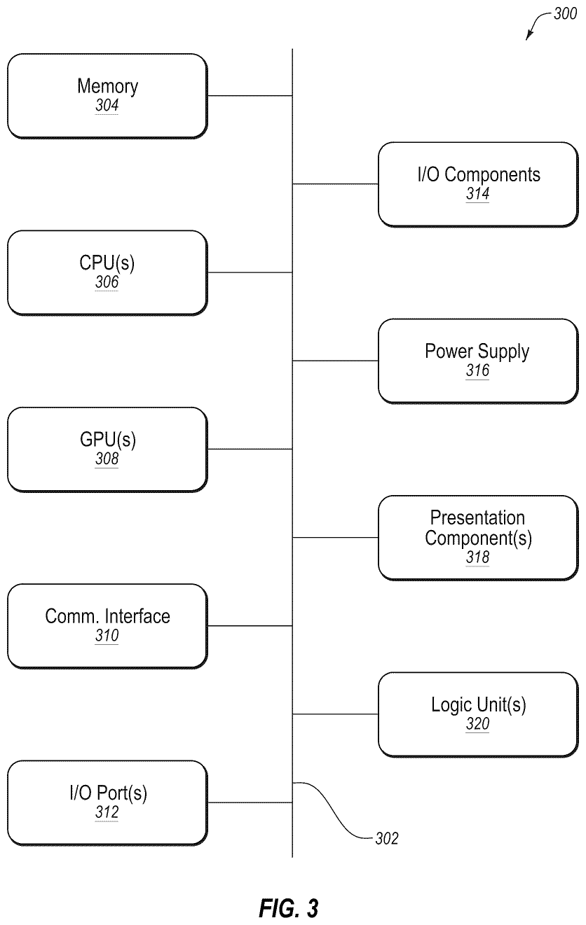

The optics module 104 may include any suitable system, apparatus, or device that may be configured to focus and direct the laser beam to the target tissue 112. For example, in some embodiments, the optics module 104 may include one or more lenses and/or one or more reflectors (e.g., mirrors). Additionally or alternatively, in some embodiments, the optics module 104 may include one or more actuators that may be configured to adjust the focusing and/or the beam direction in response to a beam control signal that may be received from the system control module 108. In these and other embodiments, the one or more actuators may be adjusted in response to a user input via any suitable user interface, such as discussed with respect to the computing system of FIG. 3. For example, in some embodiments, the user interface may include a touch screen, mouse, keyboard, joystick, foot pedal, game pad, game controller, etc., that may be used to provide commands for movement of the laser beam (e.g., via the actuators).

The imaging system 106 may include any suitable system, apparatus, or device, that may be configured to obtain one or more images of the eye corresponding to the target tissue 112. For example, in some embodiments, the imaging system 106 may collect reflected or scattered light or sound from the target tissue 112 to capture image data corresponding to the target tissue 112.

The imaging system 106 may include one or more different types of devices and/or systems configured to capture various different types of images of the eye as corresponding to the target tissue 112. For example, in some embodiments, the imaging system 106 may include a camera configured to capture one or more camera images of the eye. In these and other embodiments, the imaging system may include an ultrasound imaging device configured to capture ultrasound images of the eye.

Additionally or alternatively, the imaging system 106 may include an optical coherent tomography (OCT) device configured to capture OCT images of the eye. In these and other embodiments, the OCT images may correspond to various scans of the eye.

As indicated elsewhere in the present disclosure, OCT imaging may include splitting a beam of light into two different paths, one directed at the target tissue 112 the other toward a reference mirror. The light that reflects back from the tissue and the reference mirror is recombined to create an interference pattern, which may be analyzed to generate information about the target tissue 112. Such information may be used to generate an image of the target tissue 112.

In some embodiments, the imaging system 106 may be configured to perform multiple OCT axial scans (“A-scans”) to acquire information about the target tissue 112. An OCT A-scan may be a one-dimensional measurement that provides depth information at a single point within the target tissue 112. In particular, the A-scan may measure the time delay and intensity of light as it travels through the tissue and reflects back to a detector of the imaging system 106. The result may be a line graph that represents the reflectivity of different tissue layers along the depth axis. In some embodiments, an A-scan may be used to measure the thickness of various portions of the target tissue 112—such as the lens or other ocular structures—offering precise data about the distance from the surface of the target tissue 112 to various internal layers. In the present disclosure, reference to an “A-scan” may refer to the process of emitting and detecting light with respect to a single point of the target tissue 112, including the depth at that single point, as well as information that may be obtained from such a scan.

In these and other embodiments, the imaging system 106 may be configured to obtain one or more brightness scans (“B-scans”) that may each be based on multiple A-scans. In particular, an OCT B-scan may be a two-dimensional cross-sectional image of the target tissue 112 that may be constructed from multiple A-scans taken along a line across the target tissue 112. Each A-scan may contribute a vertical line of data, and as the scan progresses across the target tissue 112, these lines may be combined to form a detailed cross-sectional image that forms a corresponding B-scan. This image may show the various layers and structures within portions of the target tissue that are scanned, providing a more comprehensive view of the anatomy. B-scans may be used for diagnosing and monitoring conditions affecting the different portions of the eye such as the grade of cataractous lens, the cornea, the anterior segment, the retina, macula, and optic nerve, as they may reveal abnormalities in tissue structure, thickness, and reflectivity patterns across a broader area. In the present disclosure, reference to a “B-scan” may refer to the process of performing multiple A-scans as well as the cross-sectional image that may be generated from the corresponding A-scans.

In summary, a significant difference between an A-scan and a B-scan is that the A-scan provides one-dimensional depth information at a single point, while the B-scan compiles multiple A-scans to create a two-dimensional cross-sectional image. Individual A-scans may be helpful for precise measurements of tissue thickness, whereas the B-scans may offer a more comprehensive view of the structure of the target tissue 112, enabling detailed analysis and diagnosis of various conditions.

In the present disclosure, the path of the light that may be used as part of optical imaging may be referred to as “an optical measurement path.” For example, with respect to capturing camera images, the optical measurement path may refer to the path of light that passes through the lens of the camera that is detected by sensors of the camera. As another example, with respect to OCT imaging, the optical measurement path may generally refer to and include one or more paths of the light used for the OCT imaging—such as the path of the light directed toward the target tissue 112, the path of the light directed toward the reference mirror, the path of the light reflected back from the target tissue 112, and/or the path of the light reflected by the reference mirror.

The patient interface 110 may include a mount that is configured to engage with the target tissue 112 to hold the target tissue 112 in position during performance of the ophthalmic procedure. In these and other embodiments, the patient interface 110 may be configured to allow the laser beam to pass therethrough to allow for performance of the procedure via the laser beam.

In some embodiments, the patient interface 110 may include a soft-fit insert (“insert”) that may be similar to a contact lens. In these and other embodiments, the insert may be installed on an optical surface (e.g., a lens) of the patient interface 110 such that the insert may be within an optical measurement path used to perform optical imaging of the target tissue 112. For example, while the imaging system 106 is shown adjacent to the patient interface 110, it will be appreciated that the imaging system 106 may sense or otherwise image the target tissue 112 through the patient interface 110. Additionally or alternatively, the insert may be moistened (e.g., with a saline solution) to help maintain flexibility of the insert, which may be such that a liquid layer may be disposed on at least one surface of the insert.

In some instances, the liquid layer may accordingly be disposed in an optical measurement path of light that may be measured and used for optical imaging (“measurement light”). The liquid layer may also have certain refractive and/or reflective properties that may alter one or more properties of the measurement light. The altering of the measurement light may accordingly affect the analysis of such light, which may affect (e.g., distort) a resulting optical image

For example, FIG. 1B illustrates an example of measurement light 150 passing through a first layer 152 (e.g., a lens of the optics module 104 such as a soft fit insert), a liquid layer 154, and a second layer 156 (e.g., air) prior to interacting with (e.g., reflecting, scattering, etc.) the target tissue 112. As illustrated in FIG. 1B, the liquid layer 154 may have an refractive index that is different from the second layer 156 such that the liquid layer 154 may refract the measurement light 150 different than the second layer 156. Such a difference in the refraction may cause the measurement light 150 to interact with the target tissue 112 differently than if the refraction would not have occurred. In addition, the reflected measurement light 150 that reflects off (e.g., scatters off) the target tissue 112 and that is detected as part of optical imaging may have different properties due to the liquid layer 154. Further, such refraction caused by the liquid layer 154 may appear as backscattering in a corresponding B-scan. In these and other embodiments, a net result of the altering of the measurement light 150 by the liquid layer 154 may be unexpected refraction, which may be manifest as a refraction error (e.g., a difference between expected refraction and actual refraction) in the analysis of the corresponding scans. The refraction error may cause distortions in the resulting scans/images corresponding to the target tissue 112 such that the scans/images may be less accurately representative of the target tissue 112 than if the liquid layer 154 were not present. Although not illustrated in FIG. 1B, the measurement light 150 that may be reflected off of the target tissue 112 and that may be detected for the optical imaging may also be further altered as it passes back through the liquid layer 154, leading to even greater distortions.

Additionally, as illustrated in FIG. 1B, the liquid layer 154 may be non-uniform in nature. Therefore, the measurement light corresponding to different A-scans that may pass through different portions of the liquid layer 154 may be altered in a non-uniform manner such that the information between different A-scans may be distorted. Additionally or alternatively, B-scans that may be generated from the different A-scans may also be distorted in association with the distortion of the underlying A-scans.

Returning to FIG. 1A, in some instances the patient interface 110 itself may cause non-uniform refraction and/or reflection of measurement light, which may also cause distortion in corresponding A-scans and/or B-scans. For example, FIG. 1C illustrates a cross-sectional view of an example soft-fit insert 170 (“insert 170”), according to one or more embodiments of the present disclosure. As illustrated in FIG. 1C, the insert 170 may be thicker along the outside edges than toward the middle. Such non-uniformity may cause non-uniform refraction and/or reflection of measurement light with respect to different portions of the insert 170, which may also result in refraction errors that may cause distortion in corresponding A-scans and/or B-scans.

Returning to FIG. 1A, the system control module 108 may include any suitable system, apparatus, or device, configured to perform one or more control operations with respect to the system 100. For example, in some embodiments, the system control module 108 may include code and routines configured to allow a computing system to perform one or more operations. Additionally or alternatively, the system control module 108 may be implemented using hardware including one or more processors, CPUs graphics processing units (GPUs), data processing units (DPUs), parallel processing units (PPUs), microprocessors (e.g., to perform or control performance of one or more operations), field-programmable gate arrays (FPGA), application-specific integrated circuits (ASICs), accelerators (e.g., deep learning accelerators (DLAs)), one or more programmable vision accelerators (PVAs), which may include one or more vector processing units (VPUs), one or more direct memory access (DMA) systems, one or more pixel processing engines (PPEs), etc., and/or other processor types. In these and other embodiments, the control module 608 may be implemented using a combination of hardware and software. In the present disclosure, operations described as being performed by the system control module 108 may include operations that the system control module 108 may direct a corresponding computing system to perform. In these or other embodiments, the system control module 108 may be implemented by one or more computing systems, such as that described in further detail with respect to FIG. 3 of the present disclosure.

In some embodiments, the system control module 108 may be configured to control the laser 102 and/or the optics module 104. Additionally or alternatively, the system control module 108 may be configured to control any number of other components of the system 100 not expressly illustrated.

Additionally or alternatively, the system control module 108 may be configured to determine the placement of the laser beam and corresponding laser pulses with respect to the target tissue 112 in some embodiments. In these and other embodiments, the system control module 108 may be configured to determine the placement of the laser beam and the corresponding laser pulses based on one or more optical images. In these and other embodiments, the system control module 108 may be configured to determine the placement of the laser beam and the corresponding laser pulses based on an alignment registration with respect to the target tissue 112 and the one or more optical images.

Additionally or alternatively, the system control module 108 may be configured to perform one or more distortion compensation operations with respect to the optical images. Such distortion compensation operations may allow for better placement of the laser beam and corresponding laser pulses.

In some embodiments, as part of the distortion compensation, the system control module 108 may be configured to identify a non-uniform layer that is disposed in an optical measurement path (e.g., the liquid layer 154 of FIG. 1B and/or the insert 170 of FIG. 1C). In these and other embodiments, the identification of the non-uniform layer may include identifying one or more properties of the non-uniform layer. Additionally or alternatively, in some embodiments, the system control module 108 may be configured to identify the non-uniform layer based on an optical imaging result—e.g., an optical scan such as an OCT B-scan and/or an optical image such as that corresponding to an OCT B-scan.

For example, referring to FIG. 1B, backscattering in a particular OCT B-scan that may be caused by the liquid layer 154 may result in a first surface 158 and a second surface 160 being captured in the particular B-scan. In these and other embodiments, the system control module 108 may be configured to identify the liquid layer 154 based on identification of the first surface 158 and the second surface 160 (e.g., identifying a location of and/or a material of the first surface 158 and/or the second surface 160).

Additionally or alternatively, in some embodiments, the identification of the liquid layer 154 may include identifying one or more properties of the liquid layer 154. In these and other embodiments, the system control module 108 may identify the properties based on the first surface 158 and the second surface 160. For example, the system control module 108 may be configured to measure a thickness of the liquid layer 154 based on differences between the locations of the first surface 158 and the second surface 160. In these and other embodiments, the system control module 108 may be configured to measure the thickness with respect to multiple locations along the first surface 158 and the second surface 160 such that a cross-sectional shape of the liquid layer 154 may be determined by the system control module 108.

In these and other embodiments, the system control module 108 may be configured to identify one or more normal angles to the corresponding to the liquid layer 154 (e.g., angles that are perpendicular to the liquid layer 154). For example, the system control module 108 may be configured to identify one or more normal angles that are normal to (e.g., perpendicular to) the first surface 158 and/or the second surface 160. In these and other embodiments, the system control module 108 may be configured to identify multiple normal angles at multiple locations along the first surface 158 and/or the second surface 160. Additionally or alternatively, in some embodiments, the system control module 108 may be configured to identify the cross-sectional curvatures of the first surface 158 and/or of the second surface 160 based on the normal angles corresponding thereto. In these and other embodiments, the system control module 108 may be configured to identify the cross-sectional shape of the liquid layer 154 based on the curvatures of the first surface 158 and the second surface 160. In these and other embodiments, the system control module 108 may identify a cross-sectional sphericity of the liquid layer based on the curvature of the first surface 158 and/or of the second surface 160.

Additionally or alternatively, in some embodiments, the system control module 108 may be configured to determine a degree of refraction that may be caused by the liquid layer 154. For example, in some embodiments, the system control module 108 may determine that the liquid layer 154 has a particular refraction index that corresponds to saline based on an assumption that the liquid layer 154 is saline. Additionally or alternatively, the system control module 108 may determine the refractive index of the liquid layer 154 relative to other layers in the optical measurement path based on an intensity of backscattering of the measurement light that may be caused by the liquid layer 154.

Further, the system control module 108 may be configured to determine a degree of refractive that may be caused by the liquid layer 154 at any given cross-sectional location along the liquid layer 154 based on the refraction index, the normal angles corresponding to the first surface 158 and the second surface 160, respectively, at the corresponding cross-sectional location, and the thickness of the liquid layer 154 at the corresponding cross-sectional location. The degree of refraction may be determined based on any suitable optical refraction theory.

In these and other embodiments, the degree of refraction may indicate the refraction error that may be caused by the liquid layer 154 at the corresponding cross-sectional location. For example, the degree of refraction may be used to determine an overall amount of refraction that may be experienced by the measurement light which may be compared against an expected amount of refraction were the liquid layer 154 not present. The difference between the determined overall refraction and the expected refraction may indicate the refraction error.

In some embodiments, the system control module 108 may be configured to identify one or more properties of the insert 170 of FIG. 1C in a similar manner. For example, the system control module 108 may identify a first surface 172 and/or a second surface 174 of the insert 170 of FIG. 1C based on backscattering corresponding to one or more OCT B-scans in a similar manner as described with respect to the identification of the first surface 158 and the second surface 160 of the liquid layer 154. Further, a cross-sectional shape of the insert 170, normal angles of the first surface 172 and the second surface 174, cross-sectional curvatures of the first surface 172 and the second surface 174, thickness of the insert 170 at various locations, degrees of refraction caused by the insert 170, etc. may be determined in a manner similar to that described with respect to determining one or more properties of the liquid layer 154.

Returning to FIG. 1A, in some embodiments, the system control module 108 may be configured to cause performance of one or more adjustment operations with respect to one or more optical imaging results based on identification of the non-uniform layer. The adjustment operations may correspond to adjusting for distortion in the optical imaging results that may be caused by the non-uniform layer.

For example, in some embodiments, the system control module 108 may be configured to correct for distortion that may be caused by the refraction error. For instance, the system control module 108 may modify at least a portion of an optical imaging result that is identified as corresponding the non-uniform layer. Portions that correspond to the non-uniform layer may include any portion of the optical imaging result that may be affected by the non-uniform layer. For example, any portion of the optical imaging result that is distal to the non-uniform layer may be modified—e.g., portions of the optical imaging result that represent portions of the target tissue 112 that are distal to the non-uniform layer may be modified.

For instance, in some embodiments, the system control module 108 may be configured to identify portions of the optical imaging result that may be affected by the non-uniform layer—e.g., based on identifying a location of the non-uniform layer in the optical imaging result according to any suitable technique, such as based on backscattering. In these and other embodiments, the system control module 108 may be configured to determine the refraction error caused by the non-uniform layer, such as described above. Additionally or alternatively, the system control module 108 may be configured to adjust an analysis performed with respect to the portions of the optical imaging result corresponding to the non-uniform layer based on the refraction error. The adjustment may include compensating for the refraction error in the analysis according to any suitable technique in some embodiments. In these and other embodiments, the adjustment may be such that distortion included in the optical imaging result as modified may be reduced, minimized, and/or eliminated as compared to the optical imaging result as unmodified.

Additionally or alternatively, the adjustments made by the system control module 108 may include discarding one or more optical imaging results or portions of the optical imaging results that correspond to the non-uniform layer. For example, there may be instances in which the distortion may be too great to be able to adequately adjust for such distortion such that rather than attempting to modify corresponding optical imaging results, such results and/or portions corresponding to the non-uniform layer may be discarded.

In these and other embodiments, the system control module 108 may be configured to determine whether the adjustments include correcting for refraction error or discarding results based on a potential degree of distortion. For example, in some embodiments the system control module 108 may be configured to compare the determined refraction error against a refraction error threshold that may correspond to a threshold amount of distortion. In response to the determined refraction error satisfying the refraction error threshold, the system control module 108 may be configured to discard corresponding optical imaging results. Additionally or alternatively, in response to the determined refraction error not satisfying the refraction error threshold, the system control module 108 may be configured to correct for distortion in corresponding optical imaging results.

Additionally or alternatively, rather than or in addition to determining whether to correct for the distortion or discard the optical imaging results based on a determined refraction error, the system control module 108 may make such a determination based on a determined sphericity of the non-uniform layer—e.g., how spherical the non-uniform layer may be. For example, the more spherical portions of the non-uniform layer may be, the less distortion such portions may create. Conversely, the less spherical (e.g., more bulging) portions of the non-uniform layer may be, the more distortion such portions may create. As such, in some embodiments, the degree of sphericity may operate as a proxy indicator of the refraction error and/or degree of distortion. In some embodiments, the system control module 108 may accordingly determine whether to discard optical imaging results based on the degree of sphericity.

For example, in some embodiments the system control module 108 may be configured to compare the determined sphericity of a particular portion of the non-uniform layer against a sphericity threshold that may correspond to a threshold amount of distortion. In response to the determined sphericity not satisfying the sphericity threshold, the system control module 108 may be configured to discard corresponding optical imaging results. Additionally or alternatively, in response to the determined sphericity satisfying the sphericity threshold, the system control module 108 may be configured to correct for distortion in corresponding optical imaging results.

In some embodiments, the sphericity threshold and/or the refraction error threshold may be determined in any suitable manner. For example, a heuristic analysis may be performed to determine which degrees of sphericity and/or refraction error may allow for correction and which may not. Additionally or alternatively, such thresholds may vary depending on certain tolerances, computing capabilities, etc.

In these and other embodiments, the system control module 108 may be configured to cause the system 100 to modify one or more operations that are performed by the system 100 with respect to the target tissue 112 based on the one or more adjustment operations. For example, in instances in which the adjustment operations include helping correct for distortion, the system control module 108 may use the corrected optical imaging results in determining placement of the laser 102 with respect to the target tissue 112. Additionally or alternatively, in instances in which the adjustment operations include discarding certain images, the images that are retained may be used in determining placement of the laser 102. In the present disclosure, reference to placement of the laser 102 with respect to the target tissue 112 may also refer to placement of the laser beams, laser pulses, etc. that may be generated by the laser 102.

In these and other embodiments, the system control module 108 may be configured to perform one or more laser adjustment operations based on the determined laser placement. The laser adjustment operations may include any suitable operation that may move an orientation of the laser 102 and/or its corresponding laser beam with respect to the target tissue 112.

For example, in some embodiments, the system control module 108 may control one or more actuators of the optics module 104 to adjust a location of the laser beam and corresponding laser pulses. Additionally or alternatively, the system control module 108 may adjust a pattern of the laser beam through adjustment of the laser 102 and/or the optics module 104. In these and other embodiments, the system control module 108 may adjust one or more other actuators that may move the entire system 100 or the laser 102 itself such that the laser orientation may be moved with respect to the target tissue 112. Additionally or alternatively, the system control module 108 may cause the adjustment of a support platform (e.g., bed) that the patient is lying on to adjust the orientation of the laser 102 with respect to the target tissue 112 (e.g., by controlling one or more actuators corresponding to the support platform).

Modifications, additions, or omissions may be made to FIGS. 1A-1C without departing from the scope of the present disclosure. For example, the system 100 may include more or fewer elements depending on the implementation. Further, the system 100 may be configured to perform any number of operations as compared to those explicitly described. In addition, the principles described may be applied to any applicable optical imaging result and are not limited to OCT imaging. Further, the principles described may apply to any type of non-uniform layer and are not limited to liquid (e.g., saline solution) layers or soft-fit insert layers.

In addition, the use cases for adjusting for distortion may not be limited to only adjustment of a laser or other surgical device with respect to the target tissue 112. For example, the adjusting for distortion as described in the present disclosure may be applicable to any optical imaging in which a non-uniform layer may cause distortion of the optical imaging result. For instance, the detection of the distortion that may be caused by a non-uniform layer (e.g., the liquid layer 154 and/or the insert 170) may be determined whether or not a target tissue or object is present. As such, the adjusting for distortion may be applied in instances and applications that involve non-uniform layers that may be present in any suitable optical imaging technique. In some embodiments, for example, such principles may be used in calibration of, manufacturing of, maintenance of, or other uses of the system 100.

In these and other embodiments, the techniques and disclosure may relate to distortion compensation with respect to instances in which multiple non-uniform layers may be present. For example, in some instances the insert 179 may be a first non-uniform layer and the liquid layer 154 may be a second non-uniform layer. Additionally or alternatively, multiple liquid layers may be present.

In these and other embodiments, the techniques described herein may be used to determine and compensate for respective distortions that may be caused by the individual non-uniform layers. Additionally or alternatively, the respective distortions may be used to determine an aggregate amount of distortion that may be an overall distortion caused by the combination of distortions of the multiple non-uniform layers. In these and other embodiments, the distortion compensation discussed herein may be based on the aggregate distortion in some instances.

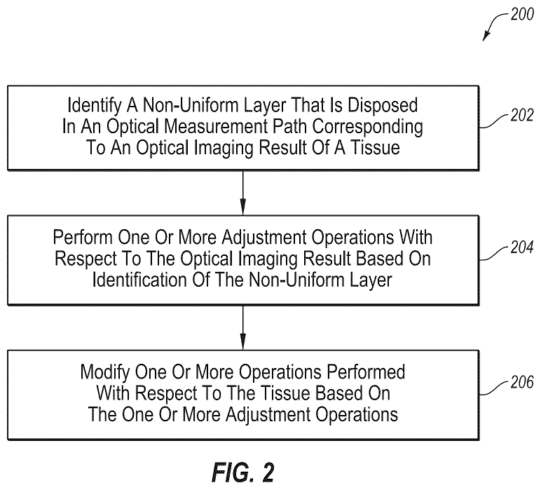

FIG. 2 is a flow diagram illustrating a method 200 for compensating for optical imaging distortion, in accordance with one or more embodiments of the present disclosure. One or more operations of the method 200 may be performed by any suitable system, apparatus, or device such as, for example, the system 100 of FIG. 1, and/or a computing system such as that described with respect to FIG. 3 of the present disclosure.

At block 202, a non-uniform layer that is disposed in an optical measurement path may be identified. In some embodiments, the optical measurement path may correspond to an optical imaging result that corresponds to a tissue, such as the target tissue 112 of FIG. 1A. In these and other embodiments, the non-uniform layer may be a liquid layer (e.g., a saline solution layer disposed on a soft-fit insert) and/or a solid layer that has non-uniform optical properties (e.g., the soft-fit insert with varying degrees of thickness that cause differences in optical refraction, reflection, etc.). In some embodiments, one or more operations described with respect to FIGS. 1A-1C that correspond to identifying a non-uniform layer may be performed with respect to block 202.

At block 204, one or more adjustment operations may be performed with respect to the optical imaging result. The adjustment operations may be based on identification of the non-uniform layer in some embodiments. Additionally or alternatively, in some embodiments, one or more operations described with respect to FIGS. 1A-1C that correspond to performing adjustment operations may be performed with respect to block 204.

At block 206, one or more operations performed with respect to the tissue may be modified. In these and other embodiments, the one or more operations may be based on the one or more adjustment operations. Additionally or alternatively, in some embodiments, one or more operations described with respect to FIGS. 1A-1C that correspond to modifying the operations may be performed with respect to block 206.

For example, in some embodiments, the one or more modification operations may include adjusting a position of an ophthalmic surgical system (e.g., adjusting positioning of laser beams generated by such a system) based on the adjustment operations, such as described above. For example, optical imaging results that have been adjusted for distortion and/or those that remain after discarding others that are deemed as having too much distortion may be used to orient laser beams with respect to the tissue (e.g., an eye) and/or a particular portion of the tissue to allow corresponding laser-based treatment tasks to be performed with respect to the tissue. In some embodiments, the adjusting of the orientation may include physically moving the positions of the laser beams—e.g., via one or more actuators or motors that move the laser itself and/or by adjusting one or more laser parameters that adjust the location of the laser beam without moving the whole laser (e.g., via adjustment of mirrors, etc.). Additionally or alternatively, the adjusting of the orientation may include changing a pattern of the laser beams. In these and other embodiments, the adjustment of the orientation may include adjusting the position of the tissue (e.g., by adjusting an orientation of a bed on which the patient is laying). In the present disclosure, reference to a “laser beam” may include any suitable beam of light that may be generated by a laser. Such light beams may be continuous or pulsed.

Modifications, additions, or omissions may be made to the method 200 without departing from the scope of the present disclosure. For example, the operations of method 200 may be implemented in differing order in some instances. Additionally or alternatively, two or more operations may be performed at the same time. Furthermore, the outlined operations and actions are only provided as examples, and some of the operations and actions may be optional, combined into fewer operations and actions, or expanded into additional operations and actions without detracting from the essence of the described embodiments.

### EXAMPLE COMPUTING SYSTEM

FIG. 3 is a block diagram of an example computing system 300 suitable for use in implementing some embodiments of the present disclosure. Computing system 300 may include an interconnect system 302 that directly or indirectly couples the following devices: memory 304, one or more central processing units (CPUs) 306, one or more graphics processing units (GPUs) 308, a communication interface 310, I/O ports 312, input/output components 314, a power supply 316, one or more presentation components 318 (e.g., display(s)), and one or more logic units 320.

Although the various blocks of FIG. 3 are shown as connected via the interconnect system 302 with lines, this is not intended to be limiting and is for clarity only. For example, in some embodiments, a presentation component 318, such as a display device, may be considered an I/O component 314 (e.g., if the display is a touch screen). As another example, the CPUs 306 and/or GPUs 308 may include memory (e.g., the memory 304 may be representative of a storage device in addition to the memory of the GPUs 308, the CPUs 306, and/or other components). In other words, the computing system of FIG. 3 is merely illustrative. Distinction is not made between such categories as “workstation,” “server,” “laptop,” “desktop,” “tablet,” “client device,” “mobile device,” “hand-held device,” “game console,” “electronic control unit (ECU),” “virtual reality system,” “augmented reality system,” and/or other device or system types, as all are contemplated within the scope of the computing system of FIG. 3.

The interconnect system 302 may represent one or more links or busses, such as an address bus, a data bus, a control bus, or a combination thereof. The interconnect system 302 may include one or more bus or link types, such as an industry standard architecture (ISA) bus, an extended industry standard architecture (EISA) bus, a video electronics standards association (VESA) bus, a peripheral component interconnect (PCI) bus, a peripheral component interconnect express (PCIe) bus, and/or another type of bus or link. In some embodiments, there are direct connections between components. As an example, the CPU 306 may be directly connected to the memory 304. Further, the CPU 306 may be directly connected to the GPU 308. Where there is direct, or point-to-point, connection between components, the interconnect system 302 may include a PCIe link to carry out the connection. In these examples, a PCI bus need not be included in the computing system 300.

The memory 304 may include any of a variety of computer-readable media. The computer-readable media may be any available media that may be accessed by the computing system 300. The computer-readable media may include both volatile and nonvolatile media, and removable and non-removable media. By way of example, and not limitation, the computer-readable media may comprise computer-storage media and communication media.

The computer-storage media may include both volatile and nonvolatile media and/or removable and non-removable media implemented in any method or technology for storage of information such as computer-readable instructions, data structures, program modules, and/or other data types. For example, the memory 304 may store computer-readable instructions (e.g., that represent a program(s) and/or a program element(s), such as an operating system. Computer-storage media may include, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that may be used to store the desired information and that may be accessed by computing system 300. As used herein, computer storage media does not comprise signals per se.

The computer storage media may embody computer-readable instructions, data structures, program modules, and/or other data types in a modulated data signal such as a carrier wave or other transport mechanism and includes any information delivery media. The term “modulated data signal” may refer to a signal that has one or more of its characteristics set or changed in such a manner as to encode information in the signal. By way of example, and not limitation, the computer storage media may include wired media such as a wired network or direct-wired connection, and wireless media such as acoustic, RF, infrared and other wireless media. Combinations of any of the above should also be included within the scope of computer-readable media.

The CPU(s) 306 may be configured to execute at least some of the computer-readable instructions to control one or more components of the computing system 300 to perform one or more of the methods and/or processes described herein. The CPU(s) 306 may each include one or more cores (e.g., one, two, four, eight, twenty-eight, seventy-two, etc.) that are capable of handling a multitude of software threads simultaneously. The CPU(s) 306 may include any type of processor, and may include different types of processors depending on the type of computing system 300 implemented (e.g., processors with fewer cores for mobile devices and processors with more cores for servers). For example, depending on the type of computing system 300, the processor may be an Advanced RISC Machines (ARM) processor implemented using Reduced Instruction Set Computing (RISC) or an x86 processor implemented using Complex Instruction Set Computing (CISC). The computing system 300 may include one or more CPUs 306 in addition to one or more microprocessors or supplementary co-processors, such as math co-processors.

In addition to or alternatively from the CPU(s) 306, the GPU(s) 308 may be configured to execute at least some of the computer-readable instructions to control one or more components of the computing system 300 to perform one or more of the methods and/or processes described herein. One or more of the GPU(s) 308 may be an integrated GPU (e.g., with one or more of the CPU(s) 306 and/or one or more of the GPU(s) 308 may be a discrete GPU. In embodiments, one or more of the GPU(s) 308 may be a coprocessor of one or more of the CPU(s) 306. The GPU(s) 308 may be used by the computing system 300 to render graphics (e.g., 3D graphics) or perform general purpose computations. For example, the GPU(s) 308 may be used for General-Purpose computing on GPUs (GPGPU). The GPU(s) 308 may include hundreds or thousands of cores that are capable of handling hundreds or thousands of software threads simultaneously. The GPU(s) 308 may generate pixel data for output images in response to rendering commands (e.g., rendering commands from the CPU(s) 306 received via a host interface). The GPU(s) 308 may include graphics memory, such as display memory, for storing pixel data or any other suitable data, such as GPGPU data. The display memory may be included as part of the memory 304. The GPU(s) 308 may include two or more GPUs operating in parallel (e.g., via a link). The link may directly connect the GPUs (e.g., using NVLINK) or may connect the GPUs through a switch (e.g., using NVSwitch). When combined together, each GPU 308 may generate pixel data or GPGPU data for different portions of an output or for different outputs (e.g., a first GPU for a first image and a second GPU for a second image). Each GPU may include its own memory, or may share memory with other GPUs.

In addition to or alternatively from the CPU(s) 306 and/or the GPU(s) 308, the logic unit(s) 320 may be configured to execute at least some of the computer-readable instructions to control one or more components of the computing system 300 to perform one or more of the methods and/or processes described herein. In embodiments, the CPU(s) 306, the GPU(s) 308, and/or the logic unit(s) 320 may discretely or jointly perform any combination of the methods, processes and/or portions thereof. One or more of the logic units 320 may be part of and/or integrated in one or more of the CPU(s) 306 and/or the GPU(s) 308 and/or one or more of the logic units 320 may be discrete components or otherwise external to the CPU(s) 306 and/or the GPU(s) 308. In embodiments, one or more of the logic units 320 may be a coprocessor of one or more of the CPU(s) 306 and/or one or more of the GPU(s) 308.

Examples of the logic unit(s) 320 include one or more processing cores and/or components thereof, such as Tensor Cores (TCs), Tensor Processing Units(TPUs), Pixel Visual Cores (PVCs), Vision Processing Units (VPUs), Graphics Processing Clusters (GPCs), Texture Processing Clusters (TPCs), Streaming Multiprocessors (SMs), Tree Traversal Units (TTUs), Artificial Intelligence Accelerators (AIAs), Deep Learning Accelerators (DLAs), Arithmetic-Logic Units (ALUs), Application-Specific Integrated Circuits (ASICs), Floating Point Units (FPUs), I/O elements, peripheral component interconnect (PCI) or peripheral component interconnect express (PCIe) elements, and/or the like.

The communication interface 310 may include one or more receivers, transmitters, and/or transceivers that enable the computing system 300 to communicate with other computing systems via an electronic communication network, including wired and/or wireless communications. The communication interface 310 may include components and functionality to enable communication over any of a number of different networks, such as wireless networks (e.g., Wi-Fi, Z-Wave, Bluetooth, Bluetooth LE, ZigBee, etc.), wired networks (e.g., communicating over Ethernet or InfiniBand), low-power wide-area networks (e.g., LoRaWAN, SigFox, etc.), and/or the Internet.

The I/O ports 312 may enable the computing system 300 to be logically coupled to other devices including the I/O components 314, the presentation component(s) 318, and/or other components, some of which may be built into (e.g., integrated in) the computing system 300. Illustrative I/O components 314 include a microphone, mouse, keyboard, joystick, game pad, game controller, satellite dish, scanner, printer, wireless device, etc. The I/O components 314 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing system 300. The computing system 300 may include depth cameras, such as stereoscopic camera systems, infrared camera systems, RGB camera systems, touchscreen technology, and combinations of these, for gesture detection and recognition. Additionally, the computing system 300 may include accelerometers or gyroscopes (e.g., as part of an inertia measurement unit (IMU)) that enable detection of motion. In some examples, the output of the accelerometers or gyroscopes may be used by the computing system 300 to render immersive augmented reality or virtual reality.

The power supply 316 may include a hard-wired power supply, a battery power supply, or a combination thereof. The power supply 316 may provide power to the computing system 300 to enable the components of the computing system 300 to operate.

The presentation component(s) 318 may include a display (e.g., a monitor, a touch screen, a television screen, a heads-up-display (HUD), other display types, or a combination thereof), speakers, and/or other presentation components. The presentation component(s) 318 may receive data from other components (e.g., the GPU(s) 308, the CPU(s) 306, etc.), and output the data (e.g., as an image, video, sound, etc.).

Modifications, additions, or omissions may be made to FIG. 3 without departing from the scope of the present disclosure. For example, the computing system 300 may include more or fewer elements depending on the implementation. Further, the computing system 300 may be configured to perform any number of operations as compared to those explicitly described.

The disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, etc., refer to codes that perform particular tasks or implement particular abstract data types. The disclosure may be practiced in a variety of system configurations, including hand-held devices, consumer electronics, general-purpose computers, more specialty computing systems, etc. The disclosure may also be practiced in distributed computing environments where tasks are performed by remote-processing devices that are linked through a communications network.

As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example The following is a non-limiting summary of some example implementations presented herein.

Example 1. A system comprising:
an optical imaging system configured to image a target tissue;
a computing system configured to cause performance of operations, the operations comprising:
   identifying, based on an optical imaging result of the optical imaging system corresponding to the target tissue, a liquid layer that is disposed in an optical measurement path between the optical imaging system and corresponding to the optical imaging result;
   performing one or more adjustment operations with respect to the optical imaging result based on identification of the liquid layer; and
   modifying one or more operations performed with respect to the tissue based on the one or more adjustment operations.

Example 2. The Example of claim 1, wherein:
the identifying of the liquid layer includes identifying one or more properties of the liquid layer; and
the one or more adjustment operations are based on at least one of the one or more properties as identified.

Example 3. The system of Example 1 or Example 2, wherein the one or more properties include at least one of:
a curvature of the liquid layer;
a sphericity of the liquid layer;
a thickness of the liquid layer;
a refractive index of the liquid layer;
a degree of refraction caused by the liquid layer; or
a refraction error caused by the liquid layer.

Example 4. The system of any of Examples 1-3, wherein the one or more adjustment operations include one or more of:
modifying a portion of the optical imaging result corresponding to the tissue; or
discarding at least the portion of the optical imaging result corresponding to the tissue.

Example 5. The system of Example 4, wherein the discarding of the optical imaging result is in response to one or more identified properties of the liquid layer indicating that a refraction error caused by the liquid layer exceeds an error threshold.

Example 6. The system of any of Examples 1-5, wherein the liquid layer is disposed on a soft-fit insert corresponding to a patient interface of an ophthalmology surgical system.

Example 7. The system of Example 6, wherein the operations further comprise performing one or more adjustment operations with respect to the optical imaging result based on the soft-fit insert in addition to the liquid layer.

Example 8. The system of any of Examples 1-7, wherein the target tissue corresponds to an eye.

Example 9. The system of any of Examples 1-8, further comprising a laser configured to emit a laser beam used to perform one or more ophthalmic treatment operations, the laser beam being adjusted based on an alignment registration.

Example 10. The system of any of Examples 1-9, further comprising a laser configured to emit a laser beam used to perform one or more ophthalmic treatment operations, wherein the modifying of the one or more operations includes adjusting an orientation of the laser beam with respect to the target tissue.

Example 11. The system of Example 10, wherein adjusting the orientation of the laser beam with respect to the target tissue includes one or more of:
adjusting a position of the laser beam with respect to the target tissue; or
adjusting a pattern of the laser beam.

Example 12. The system of Example 10 or Example 11, further comprising one or more actuators configured to adjust the orientation of the laser beam.

Example 13. The system of Example 12, wherein the one or more actuators are configured to adjust a position of the laser beam via one or more of:
adjusting one or more reflectors that reflect at least a portion of the laser beam;
adjusting an overall position of the laser; or
adjusting a position of a support on which a patient corresponding to the tissue is laying.

Example 14. The system of any of Examples 1-13, wherein the optical imaging system includes an optical coherence tomography (OCT) imaging system.

Example 15. A method performed by the system of any of claims 1-14.

What is claimed is:

1. A system comprising:
an optical imaging system configured to image a target tissue;
a computing system configured to cause performance of operations, the operations comprising:
identifying, based on an optical imaging result of the optical imaging system corresponding to the target tissue, a liquid layer that is disposed in an optical measurement path between the optical imaging system and corresponding to the optical imaging result;
performing one or more adjustment operations with respect to the optical imaging result based on identification of the liquid layer; and
modifying one or more operations performed with respect to the target tissue based on the one or more adjustment operations.

2. The system of claim 1, wherein:
the identifying of the liquid layer includes identifying one or more properties of the liquid layer; and
the one or more adjustment operations are based on at least one of the one or more properties as identified.

3. The system of claim 2, wherein the one or more properties include at least one of:
a curvature of the liquid layer;
a sphericity of the liquid layer;
a thickness of the liquid layer;
a refractive index of the liquid layer;
a degree of refraction caused by the liquid layer; or
a refraction error caused by the liquid layer.

4. The system of claim 1, wherein the one or more adjustment operations include one or more of:
modifying a portion of the optical imaging result corresponding to the target tissue; or
discarding at least the portion of the optical imaging result corresponding to the target tissue.

5. The system of claim 4, wherein the discarding of the optical imaging result is in response to one or more identified properties of the liquid layer indicating that a refraction error caused by the liquid layer exceeds an error threshold.

6. The system of claim 1, wherein the liquid layer is disposed on a soft-fit insert corresponding to a patient interface of an ophthalmology surgical system.

7. The system of claim 6, wherein the operations further comprise performing one or more adjustment operations with respect to the optical imaging result based on the soft-fit insert in addition to the liquid layer.

8. The system of claim 1, wherein the target tissue corresponds to an eye.

9. The system of claim 1, further comprising a laser configured to emit a laser beam used to perform one or more ophthalmic treatment operations, the laser beam being adjusted based on an alignment registration.

10. The system of claim 1, further comprising a laser configured to emit a laser beam used to perform one or more ophthalmic treatment operations, wherein the modifying of the one or more operations includes adjusting an orientation of the laser beam with respect to the target tissue.

11. The system of claim 10, wherein adjusting the orientation of the laser beam with respect to the target tissue includes one or more of:
adjusting a position of the laser beam with respect to the target tissue; or
adjusting a pattern of the laser beam.

12. The system of claim 10, further comprising one or more actuators configured to adjust the orientation of the laser beam.

13. The system of claim 12, wherein the one or more actuators are configured to adjust a position of the laser beam via one or more of:
adjusting one or more reflectors that reflect at least a portion of the laser beam;
adjusting an overall position of the laser; or
adjusting a position of a support on which a patient corresponding to the target tissue is laying.

14. The system of claim 1, wherein the optical imaging system includes an optical coherence tomography (OCT) imaging system.

15. A method comprising:
identifying, based on an optical imaging result corresponding to a tissue, a non-uniform layer that is disposed in an optical measurement path corresponding to the optical imaging result; and
performing one or more adjustment operations with respect to the optical imaging result based on identification of the non-uniform layer.

16. The method of claim 15, further comprising modifying one or more operations performed with respect to the tissue based on the one or more adjustment operations.

17. The method of claim 16, wherein the modifying of the one or more operations includes adjusting an orientation of a laser beam with respect to the tissue.

18. The method of claim 15, wherein the non-uniform layer includes one or more of:
a liquid layer; or
a soft-fit insert.

19. The method of claim 15, wherein:
the identifying of the non-uniform layer includes identifying one or more properties of the non-uniform layer; and
the one or more adjustment operations are based on at least one of the one or more properties as identified.

20. The method of claim 19, wherein the one or more properties include at least one of:
a curvature of the non-uniform layer;
a sphericity of the non-uniform layer;
a thickness of the non-uniform layer;
a refractive index of the non-uniform layer;
a degree of refraction caused by the non-uniform layer; or
a refraction error caused by the non-uniform layer.

21. The method of claim 15, wherein the one or more adjustment operations include one or more of:
modifying a portion of the optical imaging result corresponding to the tissue; or
discarding at least the portion of the optical imaging result corresponding to the tissue.
